(19) United States
(12) Patent Application Publication
(10) Pub. No.: US 2019/0167153 A1
(43) Pub. Date: Jun. 6, 2019

(54) EXHALATION MEASURING METHOD, EXHALATION MEASURING MODULE AND MOBILE DEVICE HAVING THE SAME

(71) Applicants: Jonathan David Piteo TARPY, Hong Kong (CN); VIRGILANT TECHNOLOGIES LIMITED, Sheung Wan (HK)

(72) Inventors: CHUN-SHENG WANG, New Taipei City (TW); JONATHAN DAVID PITEO TARPY, Taipei City (TW)

(21) Appl. No.: 16/320,482

(22) PCT Filed: Jul. 28, 2016
(86) PCT No.: PCT/US16/44420

§ 371 Date: Jan. 25, 2019

(51) Int. Cl. A61B 5/087; A61B 5/09; A61B 5/00

(52) U.S. Cl. CPC A61B 5/0871; A61B 5/09; G01F 1/00; A61B 5/6898; A61B 5/0002; A61B 5/7278

(57) ABSTRACT

The invention discloses an exhalation measuring module, which may comprise a housing, a turbine and an electromagnetic converter. The housing may be formed with a receiving space connecting to an inlet and an outlet for passing an exhalation flow from the inlet to the outlet. The turbine may comprise a plurality of magnetic vanes, which are capable of rotation under the exhalation flow. The electromagnetic converter, positioned near the turbine, may generate electrical signals representing rotational motion of the magnetic vanes. The electrical signal may be outputted by the electromagnetic converter for estimating volume of an exhalation.

### FIELD OF THE INVENTION

The present invention generally relates to an exhalation measuring method, exhalation measuring module and a mobile device having the same, specifically, the present invention relates to an exhalation measuring module with a slim size.

### BACKGROUND OF THE INVENTION

The vital capacity is one of the important clues be used to know the physiological status of a human. Because the vital capacity may differ according to the age, race, sex and so on of the human, generally, it is measured with a wet or regular spirometry and the result is evaluated by a person with medical specialty, such as a doctor, to differentiate the existence of disease. Surely, the cost is a barrier for daily measurement. Therefore, if one would like to know his/her vital capacity with acceptable cost, a hand-held spirometer which is capable to measure volume of an exhalation flow, usually of a maximal exhalation, is a good choice, because the reading is easy for people without medical specialty, and the price is affordable for common people.

### SUMMARY OF THE INVENTION

An object of the present invention is to provide an exhalation measuring method, exhalation measuring module and a mobile device having the same for measuring a characteristic of an exhalation flow of a human. Through a turbine capable to convert linear motion of the exhalation flow to rotational motion and electrical signals representing rotational motion of magnetic vanes of the turbine, the characteristic of the exhalation flow, such as volume, forced expiratory volume in the first one second (FEV1), peak expiratory flow rate (PEF), etc., preferably of a maximal exhalation, may be calculated.

In one aspect of the invention, the invention may be provided with an exhalation measuring mobile device for measuring a characteristic of an exhalation flow, comprising a platform component and a plurality of functional modules. The plurality of functional modules may be mounted on the platform component, and each of them may be configured to perform at least one function. The functional modules may comprise at least a phone call module for making a phone call and an exhalation measuring module generating a plurality of electrical signals associated to a flow rate of the exhalation flow. The exhalation measuring module may comprise a flowmeter receiving the exhalation flow, the phone call module may calculate a value for the exhalation characteristic through the electrical signals outputted from the exhalation measuring module.

In another aspect of the invention, the invention may be provided with an exhalation measuring module, which may comprise a housing, a turbine and an electromagnetic converter. The housing may be formed with a receiving space connecting to an inlet and an outlet for passing an exhalation flow from the inlet to the outlet. The turbine, positioned in the receiving space, may comprise a plurality of magnetic vanes, which are capable of rotation under the exhalation flow. The electromagnetic converter, positioned near the turbine, may generate a plurality of electrical signals representing rotational motion of the magnetic vanes. The electrical signal may be outputted by the electromagnetic converter for estimating a characteristic of the exhalation flow.

In another aspect of the invention, the invention may be provided with an exhalation measuring mobile device for measuring a characteristic of an exhalation flow, which may comprise a platform component and a plurality of functional modules. The functional modules, comprising at least a phone call module and an exhalation measuring module, may be mounted on the platform component, each of which is configured to perform at least one function. The phone call module may be for making a phone call, and the exhalation measuring module may be for converting linear motion of the exhalation flow to rotational motion. The exhalation measuring module may comprise a housing, a turbine and an electromagnetic converter. The housing may be formed with a receiving space connecting to an inlet and an outlet for passing the exhalation flow from the inlet to the outlet. The turbine, positioned in the receiving space, may comprise a plurality of magnetic vanes, which are capable of rotation under the exhalation flow. The electromagnetic converter, positioned near the turbine, may generate a plurality of electrical signals representing rotational motion of the magnetic vanes. The electrical signal may be outputted by the electromagnetic converter for estimating a characteristic of the exhalation flow.

In yet another aspect of the invention, the invention may be provided with an exhalation measuring method, which may be applied on a mobile device for measuring a characteristic of an exhalation flow. The mobile device may comprise a data receiving component, a memory storing a mobile application code and a second processing component operating to perform the mobile application code. The exhalation measuring method comprises steps of, with the data receiving component, receiving an intermediary value associated to a rotation rate of the a turbine, which converts linear motion of the exhalation flow to rotational motion, under the exhalation flow; and with the second processing component, performing the mobile application code to analyze a correlation between a flow rate and the intermediary value and calculate a value for the exhalation characteristic based on the correlation within a predetermined time period.

### BRIEF DESCRIPTION OF THE DRAWINGS

Various objects and advantages of the present invention will be more readily understood from the following detailed description when read in conjunction with the appended drawing, in which:

FIG. 1 shows a perspective view of an exhalation measuring module for measuring a characteristic of an exhalation flow according to an embodiment of the invention;

FIG. 2 shows a perspective view of the same exhalation measuring module as that of FIG. 1 in another angle;

FIG. 3 shows a perspective view of an exhalation measuring mobile device for measuring a characteristic of an exhalation flow according to an embodiment of the invention;

FIG. 4 shows a functional block of a phone call module according to an embodiment of the invention.

### DESCRIPTION OF EMBODIMENTS OF THE INVENTION

For a more complete understanding of the present disclosure and its advantages, reference is now made to the following description taken in conjunction with the accompanying drawings, in which like reference numbers indicate like features. Persons having ordinary skill in the art will understand other varieties for implementing example embodiments, including those described herein. The drawings are not limited to specific scale and similar reference numbers are used for representing similar elements. As used in the disclosures and the appended claims, the terms “example embodiment,” “exemplary embodiment,” and “present embodiment” do not necessarily refer to a single embodiment, although it may, and various example embodiments may be readily combined and interchanged, without departing from the scope or spirit of the present disclosure.

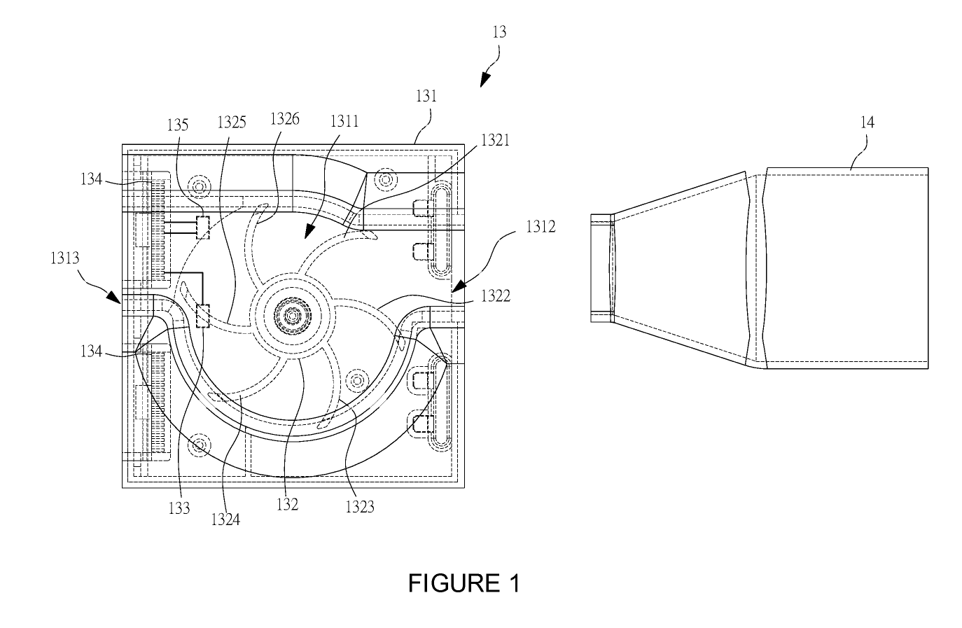

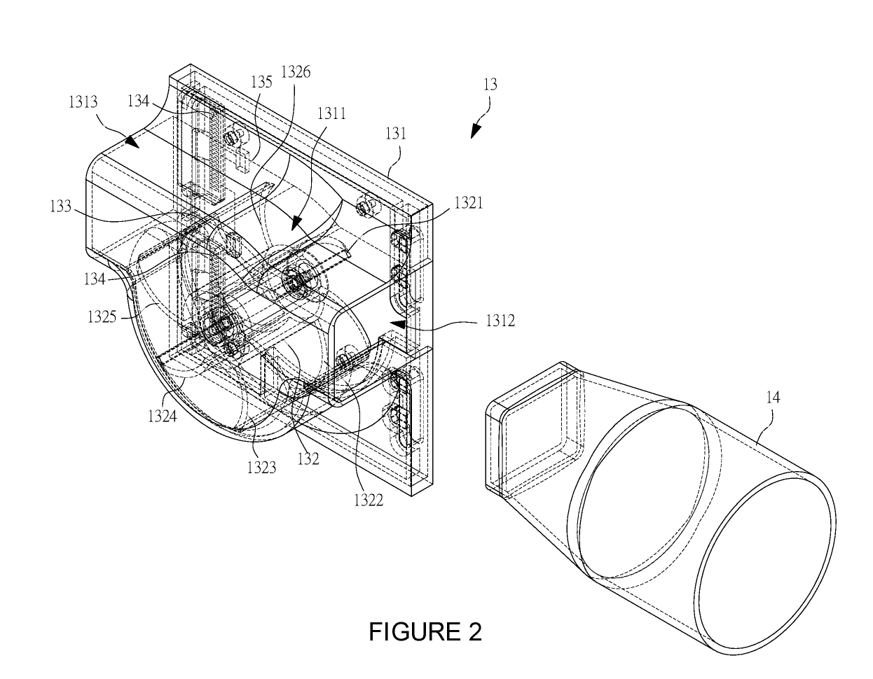

Please refer to FIGS. 1 and 2. FIG. 1 shows a perspective view of an exhalation measuring module for measuring a characteristic of an exhalation flow according to an embodiment of the invention, and FIG. 2 shows a perspective view of the same exhalation measuring module as that of FIG. 1 in another angle. At first, an exhalation measuring module 13 is utilized for converting linear motion of the exhalation flow to rotational motion. The exhalation measuring module 13 may comprise a housing 131, a turbine 132, an electromagnetic converter 133, a plurality of output terminals 134 and a first processing component 135. Preferably, the exhalation measuring module 13, with a slim size, may be adapted to configuration of a modern mobile device, which may be implemented by but not limited to a mobile phone, PDA (personal digital assistance), digital camera, watch, tablet computer, stand-alone device which is capable to perform wireless transmission, for example but not limited to that applies Wi-Fi or Bluetooth wireless communication, etc.

The housing 131 may be formed with a receiving space 1311 connecting to an inlet 1312 and an outlet 1313 for passing the exhalation flow from the inlet 1312 to the outlet 1313. For facilitating the entrance of the exhalation flow, the exhalation measuring module 13 may be paired with an air nozzle 14, which is detachedly connecting and surrounding to the inlet 1312 of the exhalation measuring module 13. The outlet 1313 is preferably larger than the inlet 1312. Please note the shape of the air nozzle 14 is only for example. The exhalation flow, preferably a maximal exhalation, may enter the receiving space 1311 from the inlet 1312 through the air nozzle 14, and then exit the receiving space 1311 from the outlet 1313.

The turbine 132, positioned in the receiving space 1311, here is formed with six magnetic vanes 1321, 1322, 1333, 1334, 1335, 1336, which are capable of rotation under the exhalation flow. The entire or a part of the turbine 132, such as the six magnetic vanes 1321, 1322, 1333, 1334, 1335, 1336, may be constructed by a magnetic material, such as iron, cobalt, nickel, the compound or alloy thereof, or a mixture of several materials including at least one magnetic material, for example but not limited to, a mixture of acrylonitrile butadiene styrene (ABS) resin and iron powder for a light weight, to serve as a permanent magnet. Here, the turbine 132. Please note that the number and shape of the magnetic vanes 1321, 1322, 1333, 1334, 1335, 1336, may be varied.

The electromagnetic converter 133 may be positioned near the turbine 132, and preferably right next to the turbine 132. Here, the electromagnetic converter 133 is implemented by a reed switch, which may be replaced by a hall effect sensor. During the rotation of the magnetic vanes 1321, 1322, 1333, 1334, 1335, 1336, due to the exhalation flow, when one of the magnetic vanes 1321, 1322, 1333, 1334, 1335, 1336, for example, the magnetic vane 1325, approaches the electromagnetic converter 133, the electromagnetic converter 133 may generate an electrical signal representing this. If it is assumed that the magnetic vanes 1321, 1322, 1333, 1334, 1335, 1336 keep rotating, another electrical signal may be generated to represent that the next magnetic vane 1336 approaches the electromagnetic converter 133, yet another electrical signal for magnetic vane 1321, and so forth. Therefore the electrical signals represent rotational motion of the magnetic vanes 1321, 1322, 1333, 1334, 1335, 1336 of the turbine 132. A part of or all the electrical signals may be outputted by the electromagnetic converter 133 for estimating the characteristic of the exhalation flow.

Then, the characteristic of the exhalation flow may be calculated through the electrical signals outputted from the electromagnetic converter 133. Here, some of the output terminals 134 may electrically connect to the electromagnetic converter 133 and some, the first processing component 135. The number of the output terminals 134 is not limited to a specific number. Through the output terminals 134, as an electrical signal-transmitting interface, the first processing component 135 may receive the electrical signals. Preferably, the first processing component 135 may be a microcontroller, microprocessor or the like which is capable to calculate an intermediary value associated to a rotation rate of the turbine 132 under exhalation flow, in average or a highest record, from the electrical signals, preferably periodically. For example, the first processing component 135 may count the number of the electrical signals received in a predetermined duration, such as a minute, 10 seconds, 1 second, 10 ms, etc. to calculate a rotation rate of the turbine 132 as the intermediary value. The intermediary value is then outputted through the output terminals 134. The characteristic of the exhalation flow may be calculated by the first processing component 135 or a separated device which receives the intermediary value based on a correlation analysis between at least one known flow and the measured rotational motion, which may be detailed later. Please note the first processing component 135 may be omitted for a simple construction in the exhalation measuring module 13; however, if so, the separated device may have more burdens on processing each electrical signal to calculate the characteristic of the exhalation flow.

The first processing component 135 or the separated device which receives the intermediary values from time to time, a series of current values of the rotation rate of the turbine 132, may analyze a correlation between at least one known flow rate curve and a curve of the rotation rate of the turbine 132, constructed by the intermediary values, preferably with assistance of a database or look-up table. In the database or look-up table, there may be associated data, such as the flow rate curves, etc., for a certain number of known flows, which may be samples from humans with different ages, races, sexes, health situations, etc. or the stored exhalation flow(s) of the user. Also, because of the mass and inertia air around the magnetic vanes 1321, 1322, 1333, 1334, 1335, 1336 here, proper corrections for the inertia of the magnetic vanes 1321, 1322, 1333, 1334, 1335, 1336 may also be implemented.

During the time period which follows the instantaneous peak flow of an exhalation, the turbine 132 may be decreasing in speed. Due to inertia, the measured rotation rate of the turbine 132, preferably in revolutions per minute (RPM), during this time period may not be representative of the actual flow. Inertia compensation may be possible by observing the rate of change of the rotation rate of the turbine 132 in addition to the instantaneous rotation rate itself. By experimentally determining the average rate at which the rotation rate of the turbine 132 decreases in speed under a no flow condition, and comparing that rate to the measured rate of change of the rotation rate of the turbine 132, the condition that the flow has stopped but the turbine 132 is spinning from inertia may be detected. Furthermore, a table correlating the rate of change of the measured rotation rate of the turbine 132 to flow during this time period may be experimentally created and stored in the first processing component 135 or the separated device. Additionally, the turbine 132 may need initial time to overcome the inertia and frictional force. An algorithm shifting the start time to a new time, an effective start time may be utilized by the first processing component 135 or the separated device for correction.

Then, the first processing component 135 or the separated device may calculate the value for the exhalation characteristic, such as volume, forced expiratory volume in the first one second (FEV1), peak expiratory flow rate (PEF), etc., based on the correlation within a predetermined time period. The FEV1, the sum of measured flow volumes within the time period of 1 second, and PEF, the absolute maximum flow, may be calculated by interpolation method with the most correlated known flow(s) to the measured flow.

In another embodiment, the first processing component may be a wireless module which is electrically connecting to the electromagnetic converter to receive the electrical signals from the electromagnetic converter, calculate and transmit the intermediary value for estimating the characteristic of the exhalation flow. In this case, both of output terminals and a first processing component are unnecessary. The wireless module may comply with wireless protocols, such as Wi-Fi or Bluetooth protocols, for wireless transmission.

In a further embodiment, preferably an implementation of a positive displacement turbine, the electrical signals generated by the electromagnetic converter may be associated to a constant volume unit defined by a volume inside the turbine or a volume of a space between two adjacent vanes for the exhalation flow. The number of the electrical signals generated corresponds to the number of the constant volume unit. The first processing component may count the number of the electrical signals received in a predetermined duration, such as a minute, 10 seconds, 1 second, etc. to calculate a count of the full rotations that the magnetic vanes rotate as the intermediary value. For example, every six electrical signals is a full rotation with the assumption of six vanes in the turbine. The correlation, analyzed by the first processing component or a separated device receiving the intermediary value, may be an algorithm constructed by parameters associated to the known volume inside the turbine or of the space between two adjacent magnetic vanes. Further, the parameters in the algorithm may be related to a diameter of the turbine, thickness of the space inside the turbine, the number of the magnetic vanes, etc. In a simple example, FEV1 of the exhalation flow may be calculated with an algorithm for calculating the count of full rotations of the turbine in a first second times the volume of the receiving space. The parameters are not limited to the examples disclosed here and may be chosen to achieve a desired accuracy for the calculation.

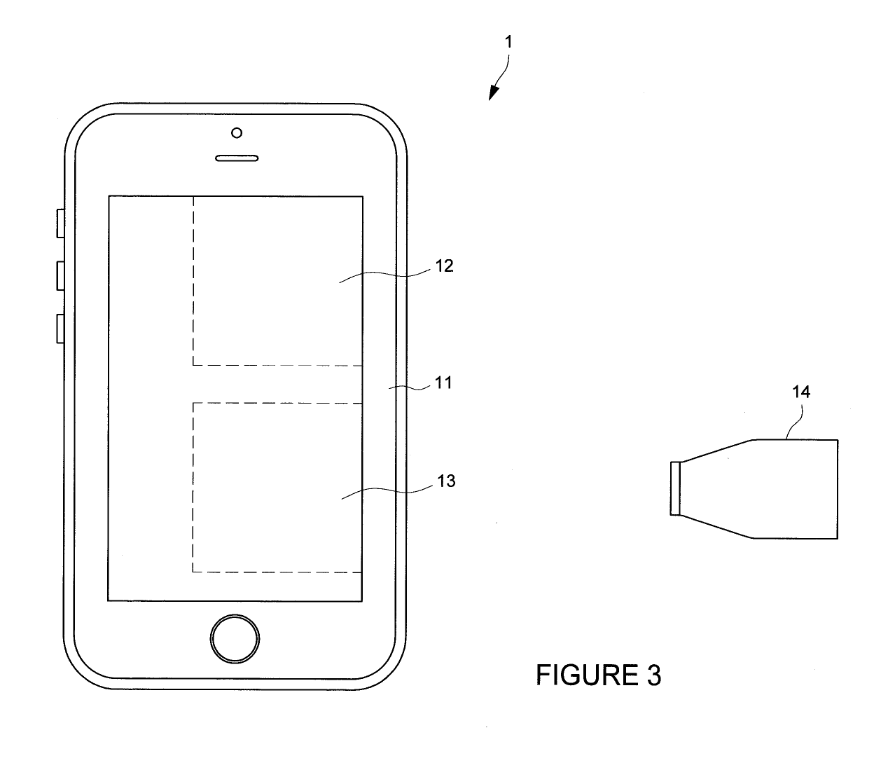

Please refer to FIG. 3, which shows a perspective view of an exhalation measuring mobile device 1 for measuring volume of an exhalation flow according to yet another embodiment of the invention. In the present embodiment, the exhalation measuring mobile device 1 is, but not limited to, a mobile phone, comprising a platform component 11 and a plurality of functional modules 12, 13, each of which is configured to perform at least one function. Specifically, the functional modules 12, 13 comprise at least a phone call module 12 for making a phone call and an exhalation measuring module 13 for converting linear motion of the exhalation flow to rotational motion, both of which may be mounted on the platform component 11. The exhalation measuring module 13 here is taking the exhalation measuring module 13 in the FIG. 1 for example. The platform component 11, the phone call module 12 and an exhalation measuring module 13 may be electrically connected to each other. Please note the number of functional modules may be varied, for example, at least one functional module with a different function may be optionally added. The platform component 11 may be detachedly connected to each functional modules 12, 13, or assembled with at least one functional modules 12, 13, such as the phone call module 12, to serve as an interface for each functional modules 12, 13 mounted thereon to communicate with each other.

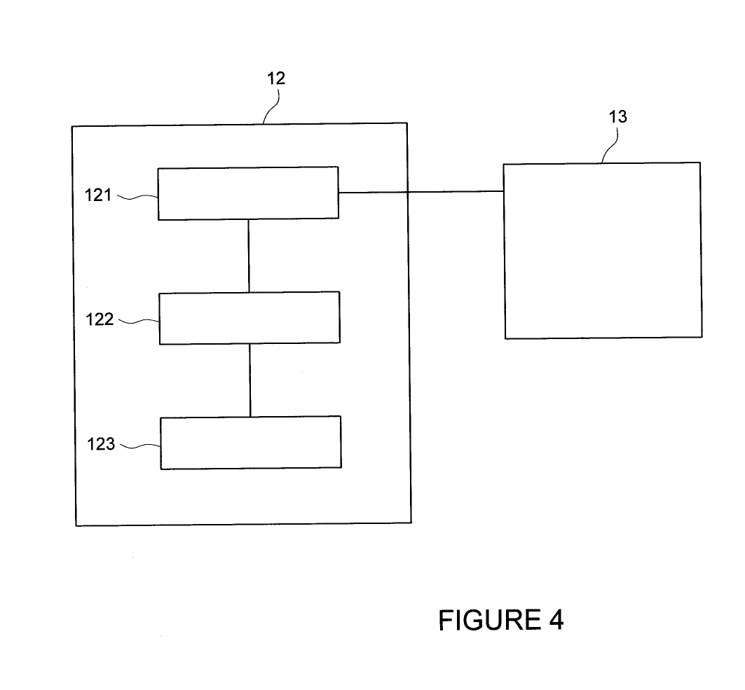

Specifically, as shown in FIG. 4, the phone call module 12 may comprise a data receiving component 121, a second processing component 122 and a memory 123. The data receiving component 121 may receive the intermediary value from the exhalation measuring module 13, such as but not limited to from the first processing component 135 or electromagnetic converter 133 shown in FIG. 1. Specifically, the data receiving component 121 may be implemented by a printed circuit board and an integrated circuit mounted thereon, a receiver complying with Bluetooth protocols, etc., depending on the type of the member passing out the intermediary value from the exhalation measuring module 13. The second processing component 122 may be electrically connecting to the data receiving component 121 and the memory 123 to control the operation of the data receiving component 121 and the memory 123.

The memory 123 may store a mobile application code, such as a mobile application. In the present embodiment, The mobile application code may be configured to, with the obedient exhalation measuring module 13, generate the electrical signals representing rotational motion of the magnetic vanes 1321, 1322, 1333, 1334, 1335, 1336, and then calculate the characteristic of the exhalation flow through the electrical signals outputted from the exhalation measuring module 13. Specifically, the second processing component 122 may execute the mobile application code to, with the first processing component 135 which receives the electrical signals, calculate the intermediary value, which is associated to a rotation rate of the turbine 132 under the exhalation flow from the electrical signals; with the data receiving component 121, receive the intermediary value from the first processing component 135; and with the second processing component 122 and the memory 123, analyze a correlation between a flow rate and the intermediary value, and calculate a value for the exhalation characteristic based on the correlation within a predetermined time period. The correlation analysis may be similar to that in the embodiment shown in FIG. 1. Therefore, as mentioned above, through the turbine capable to convert linear motion of the exhalation flow to rotational motion and the electrical signals representing rotational motion of magnetic vanes of the turbine, the characteristic of the exhalation flow may be calculated.

In another embodiment, the exhalation measuring mobile device may be implemented by a PDA (personal digital assistance), digital camera, watch, tablet computer, or stand-alone device which is capable to perform wireless transmission, for example but not limited to that applies Wi-Fi or Bluetooth wireless communication. In yet another embodiment of an exhalation measuring mobile device for measuring a characteristic of an exhalation flow, the exhalation measuring module may comprise another type of flowmeter receiving the exhalation flow instead of a turbine to generate a plurality of electrical signals associated to a flow rate of the exhalation flow. The phone call module may calculate a value for the exhalation characteristic through the electrical signals outputted from the exhalation measuring module. Please note that the flowmeter may be, but not limited to, a turbine, a differential pressure flowmeter, vortex flowmeter, ultrasonic Doppler flowmeter and the like. The differential pressure flowmeter may detect flow by measuring pressure drop across a resistance, an orifice plate, venturi tube, flow nozzle, etc. The ultrasonic Doppler flowmeter may calculate flow from ultrasonic transducers, etc.

While various embodiments in accordance with the disclosed principles been described above, it should be understood that they are presented by way of example only, and are not limiting. Thus, the breadth and scope of exemplary embodiment(s) should not be limited by any of the above-described embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, a description of a technology in the “Background” is not to be construed as an admission that technology is prior art to any invention(s) in this disclosure. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings herein.

What is claimed is:

1. An exhalation measuring module, comprising:
a housing, formed with a receiving space connecting to an inlet and an outlet for passing an exhalation flow from the inlet to the outlet;
a turbine, positioned in the receiving space, comprising a plurality of magnetic vanes which are capable of rotation under the exhalation flow; and
an electromagnetic converter, positioned near the turbine, for generating a plurality of electrical signals representing rotational motion of the magnetic vanes,
wherein the electrical signals are outputted by the electromagnetic converter for estimating a characteristic of the exhalation flow.

2. The exhalation measuring module according to claim 1, further comprising:
at least one output terminal, electrically connecting to the electromagnetic converter for transmitting the electrical signals.

3. The exhalation measuring module according to claim 1, further comprising:
a first processing component, receiving the electrical signals and calculating at least one intermediary value associated to a rotation rate of the turbine under the exhalation flow from the electrical signals.

4. The exhalation measuring module according to claim 3, wherein the first processing component is a microcontroller.

5. The exhalation measuring module according to claim 1, further comprising:
a wireless module, electrically connecting to the electromagnetic converter, receiving the electrical signals from the electromagnetic converter, calculating and transmitting an intermediary value associated to a rotation rate of the turbine under exhalation flow.

6. The exhalation measuring module according to claim 5, wherein the wireless module complies with Bluetooth protocols.

7. An exhalation measuring mobile device for measuring a characteristic of an exhalation flow, comprising:
a platform component; and
a plurality of functional modules, mounted on the platform component, each of which configured to perform at least one function, comprising at least a phone call module for making a phone call and an exhalation measuring module for converting linear motion of the exhalation flow to rotational motion, comprising:
a housing, formed with a receiving space connecting to an inlet and an outlet for passing the exhalation flow from the inlet to the outlet;
a turbine, positioned in the receiving space, comprising a plurality of magnetic vanes which are capable of rotation under the exhalation flow; and
an electromagnetic converter, positioned near the turbine, for generating a plurality of electrical signals representing rotational motion of the magnetic vanes,
wherein the phone call module calculates a value for the exhalation characteristic through the electrical signals outputted from the electromagnetic converter.

8. The exhalation measuring mobile device according to claim 7, wherein the exhalation measuring module further comprises:
a first processing component, receiving the electrical signals and calculating at least one intermediary value associated to a rotation rate of the turbine under the exhalation flow from the electrical signals.

9. The exhalation measuring mobile device according to claim 8, wherein the first processing component is a microcontroller.

10. The exhalation measuring mobile device according to claim 8, wherein the phone call module comprises:
a data receiving component, receiving the intermediary value from the first processing component;
a memory, storing a mobile application code; and
a second processing component, electrically connecting to the data receiving component and the memory, operating to perform the mobile application code;
wherein the mobile application code is configured to, with the data receiving component, the memory and the second processing component, cause the exhalation measuring mobile device to at least:
analyze a correlation between a flow rate and the intermediary value; and
calculate the value for the exhalation characteristic based on the correlation within a predetermined time period.

11. The exhalation measuring mobile device according to claim 10, wherein the mobile application code is configured to, with the data receiving component, the memory and the second processing component, cause the exhalation measuring mobile device to further:
calculate a peak expiratory flow rate of the exhalation flow based on the correlation.

12. The exhalation measuring mobile device according to claim 10, wherein the data-receiving component is a printed circuit board and an integrated circuit mounted thereon.

13. The exhalation measuring mobile device according to claim 7, wherein the exhalation measuring module further comprises:
a wireless module, electrically connecting to the electromagnetic converter, receiving the electrical signals from the electromagnetic converter, calculating and transmitting an intermediary value associated to a rotation rate of the turbine under exhalation flow.

14. The exhalation measuring mobile device according to claim 13, wherein both the wireless module and the data-receiving component comply with Bluetooth protocols.

15. The exhalation measuring mobile device according to claim 7, further comprising:
an air nozzle, detachedly connecting and surrounding to the inlet of the exhalation measuring module.

16. An exhalation measuring method for measuring a characteristic of an exhalation flow, applied in a mobile device comprising a data receiving component, a memory storing a mobile application code and a second processing component operating to perform the mobile application code, comprising:
with the data receiving component, receiving an intermediary value associated to a rotation rate of the a turbine, which converts linear motion of the exhalation flow to rotational motion, under the exhalation flow; and
with the second processing component, performing the mobile application code to analyze a correlation between a flow rate and the intermediary value and calculate a value for the exhalation characteristic based on the correlation within a predetermined time period.

17. The exhalation measuring method according to claim 16, further comprising:
calculating a peak expiratory flow rate of the exhalation flow based on the correlation.

18. An exhalation measuring mobile device for measuring a characteristic of an exhalation flow, comprising:
a platform component; and
a plurality of functional modules, mounted on the platform component, each of which is configured to perform at least one function, comprising at least a phone call module for making a phone call and an exhalation measuring module, comprising a flowmeter receiving the exhalation flow, generating a plurality of electrical signals associated to a flow rate of the exhalation flow,
wherein the phone call module calculates a value for the exhalation characteristic through the electrical signals outputted from the exhalation measuring module.

19. The exhalation measuring mobile device according to claim 18, wherein the flowmeter is a turbine, a differential pressure flowmeter, a vortex flowmeter, or an ultrasonic Doppler flowmeter.
